(19) United States
(12) Patent Application Publication
(10) Pub. No.: US 2024/0065828 A1
(43) Pub. Date: Feb. 29, 2024

(54) ADAPTIVE INTRAOCULAR LENS HOLDER

(71) Applicant: Johnson & Johnson Surgical Vision, Inc., Irvine, CA (US)

(72) Inventors: Yasemar Perez, Aguadilla, PR (US); Scott Ansell, Jacksonville, FL (US)

(21) Appl. No.: 18/458,764

(22) Filed: Aug. 30, 2023

Related U.S. Application Data
(60) Provisional application No. 63/374,235, filed on Aug. 31, 2022.

(51) Int. Cl. A61F 2/16

(52) U.S. Cl. CPC A61F 2/1691

(57) ABSTRACT

Disclosed herein is a one-piece intraocular lens holder that may contain an interocular lens. The one-piece holder comprises a first portion, a second portion, and at least one integral hinge that connects the first portion to the second portion, wherein one portion comprises an integral annular support that accommodates an intraocular lens and the other portion comprises an integral adaptive feature that exerts a force on the intraocular lens when the intraocular lens is positioned on the integral annular support and the two portions are in a closed position.

### CROSS REFERENCE TO RELATED APPLICATIONS

This application claims priority to U.S. Provisional Application No. 63/374,235, filed on Aug. 31, 2022, which is hereby incorporated by reference in its entirety.

### FIELD OF TECHNOLOGY

The field of the disclosure relates generally to an intraocular lens (IOL) holder. More particularly, the disclosure relates to a holder, case, cassette, and/or package for holding, transporting, shipping, carrying, washing, sterilizing, aerating, degassing, and/or storing IOLs of various types in a variety of models, shapes, and sizes.

### BACKGROUND

Artificial IOLs can be used to replace the natural lens of the eye, e.g., for the treatment of cataracts, or can be used to correct vision while leaving the natural lens of the eye in place. There are several types of artificial IOLs that are commonly used in eye surgery that are available in a variety of models, shapes, and sizes, and that are manufactured from various materials.

Intraocular lenses are made from various biocompatible materials including, but not limited to, polymethylmethacrylate (PMMA), silicone polymers, acrylic polymers, hydrogel polymers, and the like. Different types of IOLs include, but are not limited to, monofocal, multifocal, accommodating, presbyopic-correcting, and toric lens implants. In addition to the lens body, commonly referred to as the optic, most IOLs also include haptic features that are primarily responsible for positioning and maintaining mechanical and rotational stability of the IOL implant in the eye. Intraocular lenses can be manufactured as single piece lenses, which are manufactured with the optic and the haptics together from the same material, or as multiple piece lenses, wherein the optic and the haptics may be made of different materials. The size, shape, and thickness of the optic as well as the size, shape and number of lens haptics varies among different types and models of intraocular lens.

Along with the development of many different types, models, shapes, and sizes of intraocular lenses being manufactured from various materials, various shapes, sizes, and designs of IOL holders have been developed. IOL holders carry out a variety of functions including, but not limited to, providing a means of protection and support for holding, transporting, shipping, carrying, washing, sterilizing, aerating, degassing, and storing IOLs.

Currently, most IOL holders are composed of several different materials; require multiple pieces and/or different inserts or parts to accommodate different types, models, shapes, and sizes of IOLs; and/or require specialized assembly, often required to be completed at manufacturing sites, which adds complexity to the components supply chain and manufacturing operations. A more universal IOL holder is needed that is adaptive to accommodate different types, models, shapes, and sizes of IOLs without requiring multiple components or specialized assembly.

### BRIEF SUMMARY

In one aspect, this disclosure relates to an IOL holder comprising a one-piece device comprising a base, a lid, and at least one integral hinge that connects the base to the lid, wherein the base comprises an integral annular support that accommodates an intraocular lens, wherein the lid comprises an integral adaptive feature that exerts a force on a lens body of an intraocular lens when an intraocular lens is positioned on the integral annular support of the base and the lid is in a closed position.

In another aspect, this disclosure relates to a method of manufacture of an IOL holder comprising forming an IOL holder as a one-piece device from at least one polymer, wherein the IOL holder comprises a one-piece device comprising a base, a lid, and at least one integral hinge that connects the base to the lid, wherein the base of the IOL holder comprises an integral annular support that is designed to accommodate an intraocular lens, and wherein the lid of the IOL holder comprises an integral adaptive feature that exerts a force on a lens body of an intraocular lens when an intraocular lens is positioned on the integral annular support of the base and the lid is in a closed position.

In another aspect, this disclosure relates to a method of use of an IOL holder comprising holding, transporting, shipping, carrying, washing, sterilizing, aerating, degassing, and/or storing an intraocular lens in an IOL holder, wherein the IOL holder comprises a one-piece device comprising a base, a lid, and at least one integral hinge that connects the base to the lid, wherein the base of the IOL holder comprises an integral annular support that is designed to accommodate an intraocular lens, and wherein the lid of the IOL holder comprises an integral adaptive feature that exerts a force on a lens body of an intraocular lens when an intraocular lens is positioned on the integral annular support of the base and the lid is in a closed position.

### BRIEF DESCRIPTION OF THE DRAWINGS

The Figures described herein depict primarily generalized embodiments or data in support of generalized embodiments, which embodiments will be described with additional specificity and detail in connection with the drawings in which:

FIG. 1 is an illustration of an embodiment of the IOL holder disclosed herein displayed with a lid in an open position with a single piece intraocular lens in place on the integral annular support with the intraocular lens haptics in contact with the recess features of the integral posts.

FIG. 2 is an illustration of an embodiment of the IOL holder disclosed herein displayed with a lid in an open position with a multiple piece (3-piece) intraocular lens in place on the integral annular support with the intraocular lens haptics partially wrapped around the integral posts.

FIG. 3 is an illustration of an embodiment of the IOL holder disclosed herein displayed with a lid in a closed position.

FIG. 4 is an illustration of an embodiment of the IOL holder disclosed herein displayed with a lid in an open position with a single piece intraocular lens in place on the integral annular support with the intraocular lens haptics in contact with the recess features of the integral posts.

FIG. 5 is an illustration of an embodiment of the IOL holder disclosed herein displayed with a lid in a closed position.

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified unless the context clearly dictates otherwise. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “IOL holder” refers to any device that is designed to be used for one or more function including, but not limited to, holding, transporting, shipping, carrying, washing, sterilizing, aerating, degassing, and storing IOLs of various types, models, shapes, and sizes. The term “holder” may be used interchangeably with case, cassette, package, and related terms that refer to similar devices that perform similar functions.

As used herein, the terms “intraocular lens” and “IOL” refer to an artificial lens that is designed to be placed within the eye of a subject. The IOL includes both the lens body, i.e., the optic, and may also include haptic features. Intraocular lenses are made from various materials including, but not limited to, thermal plastic, polymethylmethacrylate (PMMA), silicone, and hydrophobic acrylic. Different types of IOLs include, but are not limited to, monofocal, multifocal, accommodating, presbyopic-correcting, and toric lens implants. Intraocular lenses can be manufactured as single piece lenses, which are manufactured with the optic and the haptics together from the same material, or as multiple piece lenses, wherein the optic and the haptics may be made of different materials. The size, shape, and thickness of the optic as well as the size, shape and number of lens haptics varies among different types and models of intraocular lens.

As used herein, the terms “lens body,” and “optic” specifically refer to the portion of an IOL that is designed to refract light.

As used herein, the terms “haptic features” and “haptics” refer to the portion of an IOL that is specifically designed for positioning and maintaining mechanical and rotational stability of the IOL implant in the eye. Intraocular lenses may have one or more haptics, and the haptics may be manufactured from the same material as the optic, e.g., in single piece lenses, or the haptics may be made from a material that is different from the optic, e.g., for most multiple piece lenses. Each haptic may be joined with the lens at one connection, or at two with so-called loop-type haptics.

As used herein, the terms “using” refers to such methods of use comprising holding, transporting, shipping, carrying, washing, sterilizing, aerating, degassing, and/or storing, and the like.

As used herein, the term “subject” refers to a warm-blooded animal such as a mammal, which is to benefit from the replacement or insertion of at least one lens in the eye with an intraocular lens. It is understood that at least humans, dogs, cats, and horses are within the scope of the meaning of the term.

The present application discloses a one-piece IOL holder that is surprisingly adaptive to accommodate different types, models, shapes, and sizes of IOLs without requiring multiple parts or complex assembly. Further disclosed is a method of manufacture of an IOL holder that is surprisingly adaptive to accommodate different types, models, shapes, and sizes of IOLs. Further disclosed is a method of use of an IOL holder that is surprisingly adaptive to accommodate different types, models, shapes, and sizes of IOLs. The IOL holder, method of manufacture of the IOL holder, and method of use of the IOL holder disclosed herein provide benefits over the state of the art by reducing manufacturing complexities and costs, while increasing flexibility, efficiency, and effectiveness.

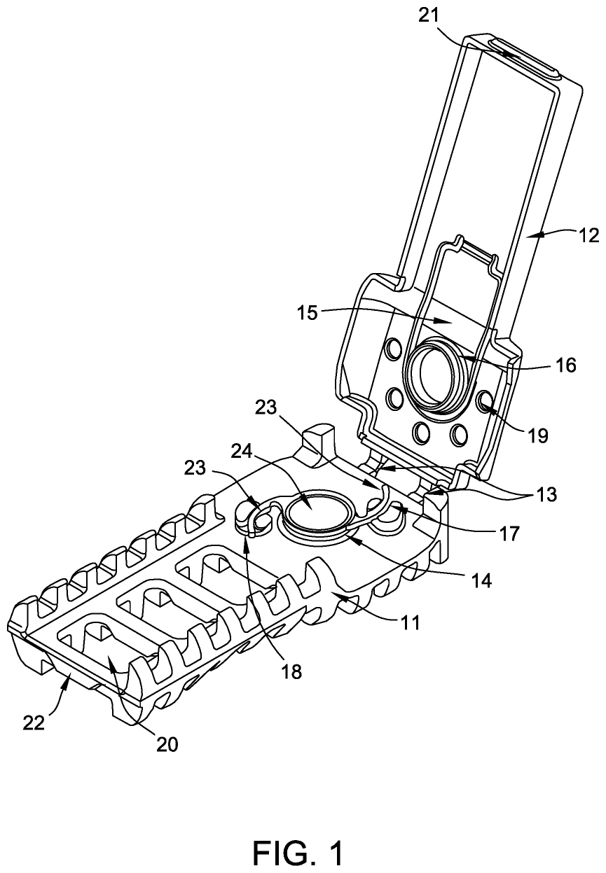

In various embodiments, the IOL holder comprises a one-piece device comprising a base, a lid, and at least one integral hinge that connects the base to the lid, wherein the base of the IOL holder comprises an integral annular support that is designed to accommodate an intraocular lens, and wherein the lid of the IOL holder comprises an integral adaptive feature that exerts a force on a lens body of an intraocular lens when an intraocular lens is positioned on the integral annular support of the base and the lid is in a closed position. Preferentially, as depicted in FIG. 1, the base 11 and the lid 12 of the IOL holder are integrally connected by two integral hinges 13.

In various embodiments, the IOL holder is manufactured from a polymer. Suitable polymers include those that are compatible with the material of construction of an intraocular lens and the methods of use of the IOL holder including, but not limited to, thermal plastics, biopolymers, and copolymers. More specifically, the polymers include, but are not limited to, polyolefins (e.g., polypropylene and polyethylene), polycarbonate, acrylic polymers, acrylic copolymers, nylon, polyester, cellulose acetate, acetate/butyrate, and the like. In one embodiment, a glassomer insert may be used. Preferably, the IOL holder comprises polypropylene or a thermal plastic.

In various embodiments, the IOL holder can accommodate various types, models, shapes, and sizes of a single piece or a multiple piece intraocular lens. In various embodiments, the IOL holder can accommodate various types, models, shapes, and sizes of a single piece intraocular lens. In various embodiments, the IOL holder can accommodate various types, models, shapes, and sizes of a multiple piece intraocular lens.

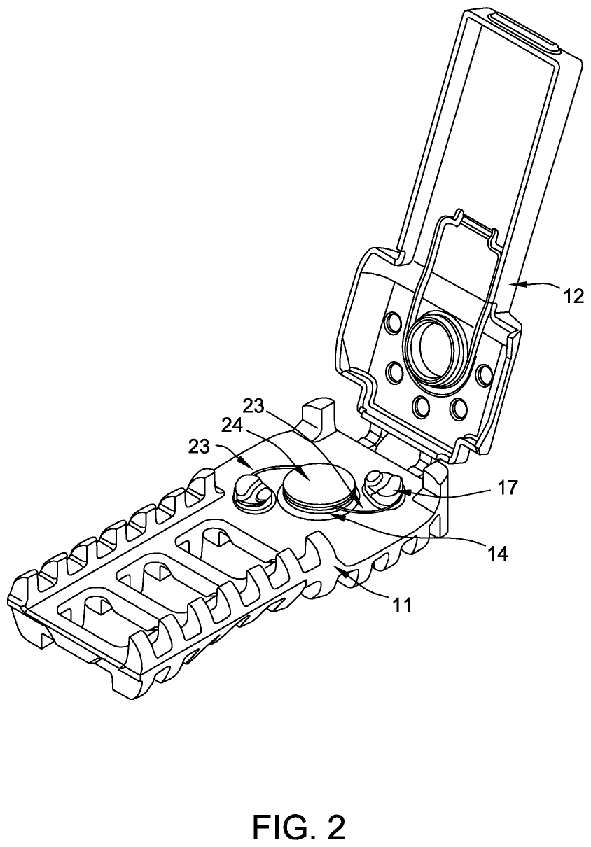

In various embodiments, the integral annular support is designed to protrude from the base of the IOL holder as a column comprising a concave profile at the distal end of the integral annular support with respect to the base, such that an intraocular lens may be positioned on the concave profile. In various embodiments, the integral annular support is designed to be level with the base of the IOL holder and comprise a concave profile in which an intraocular lens may be positioned. In various embodiments, the integral annular support is designed to recede within the base of the IOL holder and comprise a concave profile in which an intraocular lens may be positioned. In the device depicted in FIGS. 1 and 2, the integral annular support 14 protrudes from the base 11 in a columnar shape and comprises a concave profile at the distal end of the integral annular support 14 with respect to the base 11.

It will be appreciated that the integral annular support 14 does not have to have a columnar shape base portion. The base of the support may be of any shape. By way of example, and not of limitation, the base of the integral annular support 14 may be oval, square, rectangular, pentagonal, hexagonal, etc. However, in one aspect of the invention the portion which contacts the optic portion of the intraocular lens will be annular in shape.

In various embodiments, the integral annular support 14 is designed to avoid significant contact with the optical surface of the lens. In further embodiments, the integral annular support is designed to avoid contact with the optical surface of the lens. In still further embodiments, the integral annular support is designed to avoid contact with the central portion of the optical surface of the lens within a 5 mm diameter. In still further embodiments, the integral annular support is designed to avoid contact with the central portion of the optical surface of the lens within a 3 mm diameter.

In various embodiments, the integral annular support is designed to avoid significant contact with the optical surface of the lens. In further embodiments, the integral annular support is designed to avoid contact with the optical surface of the lens. In still further embodiments, the integral annular support is designed to avoid contact with the central portion of the optical surface of the lens within a 5 mm diameter. In still further embodiments, the integral annular support is designed to avoid contact with the central portion of the optical surface of the lens within a 3 mm diameter.

In various embodiments, the IOL holder includes an integral adaptive feature that comprises a cantilever design, wherein only one end of the integral adaptive feature is integrally connected to the lid. In various embodiments, the IOL holder includes an integral adaptive feature that comprises a cantilever design, wherein the integral adaptive feature is capable of being positioned at angles inward from, outward from, and/or level with the lid along the length of the integral adaptive feature. In various embodiments, the IOL holder includes an integral adaptive feature that is integrally connected to the lid by one or more flexible connections.

In various embodiments, the angle of the integral adaptive feature with respect to the lid is set during manufacturing such that the integral adaptive feature will exert a desired force on a lens body of an intraocular lens when an intraocular lens is positioned on the integral annular support of the base and a lid is in a closed position. In various embodiments, the length of the integral adaptive feature with respect to the lid is set during manufacturing such that the integral adaptive feature will exert a desired force on a lens body of an intraocular lens when an intraocular lens is positioned on the integral annular support of the base and a lid is in a closed position. In various embodiments, the width of the integral adaptive feature with respect to the lid is set during manufacturing such that the integral adaptive feature will exert a desired force on a lens body of an intraocular lens when an intraocular lens is positioned on the integral annular support of the base and a lid is in a closed position. In various embodiments, the thickness of the of the integral connection of the integral adaptive feature with to the lid is set during manufacturing such that the integral adaptive feature will exert a desired force on a lens body of an intraocular lens when an intraocular lens is positioned on the integral annular support of the base and a lid is in a closed position.

As one skilled in the art will appreciate, the design of the connection of the integral adaptive feature to the IOL holder will influence the force that is exerted on the lens body. In the embodiment of the cantilever design, fulcrum width, fulcrum thickness and lever length will influence force. Material selection and thickness of the connection(s) will also influence force.

In various embodiments, the IOL holder includes an integral adaptive feature that comprises an integral annular protrusion that exerts a force on a lens body of an intraocular lens when an intraocular lens is positioned on the integral annular support of the base and a lid is in a closed position. In various embodiments, the IOL holder includes an integral adaptive feature that comprises an integral annular protrusion that comprises a concave profile and is positioned to apply a desired force on an intraocular lens when an intraocular lens is placed in the integrated annular support of the base and the lid is in a closed position.

In various embodiments, the IOL holder includes an integral adaptive feature that comprises an integral annular recession that exerts a force on a lens body of an intraocular lens when an intraocular lens is positioned on the integral annular support of the base and a lid is in a closed position. In various embodiments, the IOL holder includes an integral adaptive feature that comprises an integral annular recession that comprises a concave profile and is positioned to apply a desired force on an intraocular lens when an intraocular lens is placed in the integrated annular support of the base and the lid is in a closed position.

In various embodiments, the IOL holder includes an integral adaptive feature that comprises an integral annular feature that is level with the lid and that exerts a force on a lens body of an intraocular lens when an intraocular lens is positioned on the integral annular support of the base and a lid is in a closed position. In various embodiments, the IOL holder includes an integral adaptive feature that comprises an integral annular feature that is level with the lid and that comprises a concave profile and is positioned to apply a desired force on an intraocular lens when an intraocular lens is placed in the integrated annular support of the base and the lid is in a closed position.

In various embodiments, the IOL holder includes an integral adaptive feature that exerts a force on a lens body of an intraocular lens when an intraocular lens is positioned on the integral annular support of the base and a lid is in a closed position, wherein the force is enough to hold an intraocular lens in place on an integral annular support of a base without causing damage to an intraocular lens.

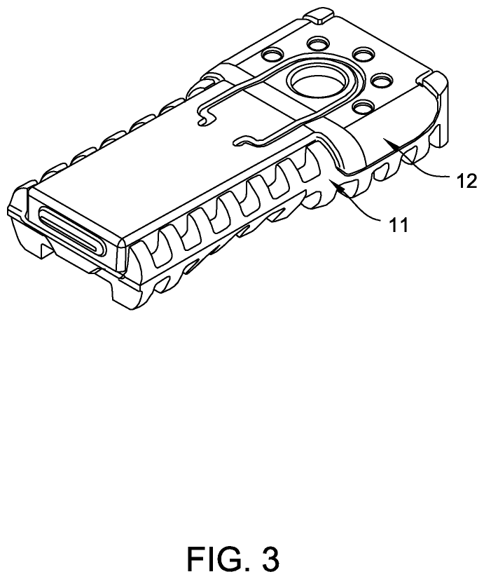

In various embodiments, as depicted in FIGS. 1 and 3, the IOL holder includes an integral adaptive feature 15 that comprises an integral annular protrusion 16 that applies a desired force on an intraocular lens when an intraocular lens is placed in the integrated annular support 14 of the base 11 and the lid 12 is in a closed position such that the force is enough to hold an intraocular lens 24 in place on an integral annular support of a base without causing damage to an intraocular lens 24.

In various embodiments, the integral annular protrusion is designed to avoid significant contact with the optical surface of the lens. In further embodiments, the integral annular protrusion is designed to avoid contact with the optical surface of the lens. In still further embodiments, the integral annular protrusion is designed to avoid contact with the central portion of the optical surface of the lens within a 5 mm diameter. In still further embodiments, the integral annular protrusion is designed to avoid contact with the central portion of the optical surface of the lens within a 3 mm diameter.

In various embodiments, the IOL holder includes a base that comprises one or more integral posts positioned in proximity to an integral annular support of the base. In various embodiments the IOL holder includes one or more integral posts positioned at a distance from the integral annular support such that the haptics of an intraocular lens placed on the integrated annular support can make contact with the one or more integral posts.

In various embodiments, the IOL holder includes a base that comprises one or more integral posts positioned in proximity to an integral annular support of the base, wherein the one or more integral posts provide one or more contact points for one or more haptics when an intraocular lens is positioned on the integral annular support of the base. In various embodiments the haptics of an intraocular lens placed on the integrated annular support can make contact with the top of the one or more integral posts. In various embodiments the haptics of an intraocular lens placed on the integrated annular support can make contact with the sides of the one or more integral posts. In various embodiments the haptics of an intraocular lens placed on the integrated annular support can wrap at least partially around the sides of the one or more integral posts.

In various embodiments, the IOL holder includes a base that comprises one or more integral posts positioned in proximity to an integral annular support of the base, wherein the one or more integral posts comprise at least one recess feature. In various embodiments, the at least one recess feature is positioned on the top of the one or more integral posts. In various embodiments, the at least one recess feature is positioned on the side of the one or more integral posts.

In various embodiments, the IOL holder includes a base that comprises one or more integral posts positioned in proximity to an integral annular support of the base, wherein the one or more integral posts comprise at least one recess feature, wherein the at least one recess feature of the one or more integral posts provides one or more contact points for one or more haptics when an intraocular lens is positioned on the integral annular support of the base. In various embodiments, the haptics of an intraocular lens placed on the integrated annular support can make contact with the at least one recess feature that is positioned on the top of the one or more integral posts. In various embodiments, the haptics of an intraocular lens placed on the integrated annular support can make contact with the at least one recess feature that is positioned on the side of the one or more integral posts. In various embodiments, the haptics of an intraocular lens placed on the integrated annular support can make contact with the at least one recess feature that is positioned on the side of the one or more integral posts when the haptics wrap at least partially around the sides of the one or more integral posts.

In an embodiment of the invention depicted in FIG. 1, the base 11 of the IOL holder comprises two integral posts 17 that each include at least one recess feature 18, wherein the integral posts 17 are positioned at a distance from the integral annular support 14 of the base 11 such that the haptics 23 of a intraocular lens 24 placed on the integrated annular support 14 can make contact with the at least one recess feature 18 that is positioned on the top of each of the integral posts 17.

An intraocular lens associated with an aspect of the invention may include a single piece intraocular lens or a three-piece intraocular lens, by way of non-limiting example.

In various embodiments, the IOL holder includes a lid that comprises one or more apertures to allow gas and/or liquid to flow through to an intraocular lens when an intraocular lens is positioned on the integral annular support of the base and the lid is in the closed position. In various embodiments, the one or more apertures included in the lid are arranged in a semicircular pattern positioned around the portion of the lid that is in close proximity to the end of the integral adaptive feature that exerts a force on a lens body of an intraocular lens when an intraocular lens is positioned on the integral annular support of the base and the lid is in a closed position. In various embodiments, the one or more apertures included in the lid are arranged in a linear pattern. In various embodiments, the one or more apertures included in the lid are arranged in a partial square or rectangular pattern. In various embodiments, the one or more apertures included in the lid are each circular, ovular, square, rectangular, or triangular in shape. In the embodiment of the invention shown in FIG. 1, the one or more apertures 19 included in the lid 12 are circular in shape and arranged in a semicircular pattern.

In various embodiments, the IOL holder includes a base that comprises one or more apertures to allow gas and/or liquid to flow through to an intraocular lens when an intraocular lens is positioned on the integral annular support of the base and the lid is in the closed position. In various embodiments, the one or more apertures included in the base are arranged in a linear pattern aligned from the end distal to the integral annular support and leading towards, but short of the integral annular support. In various embodiments, the one or more apertures included in the base are arranged in a diagonal pattern. In various embodiments, the one or more apertures included in the base are arranged in a circular, ovular, square, rectangular, or triangular pattern. In various embodiments, the one or more apertures included in the base are each circular, ovular, square, rectangular, or triangular in shape. In FIG. 1, the one or more apertures 20 included in the base 11 are rectangular in shape and arranged in a linear pattern.

In various embodiments, the IOL holder includes a lid that automatically interlocks with a base when the lid is in the closed position, wherein the interlocking of the lid and the base is reversible. In various embodiments, the IOL holder includes a lid that automatically interlocks with a base when the lid is in the closed position, wherein the interlocking of the lid and the base is reversible, wherein the lid comprises an interlocking recess, wherein the base comprises an interlocking protrusion, and wherein the interlocking recess and the interlocking protrusion provide a means for the interlocking of the lid and the base. In various embodiments, the IOL holder includes a lid that automatically interlocks with a base when the lid is in the closed position, wherein the interlocking of the lid and the base is reversible, wherein the lid comprises an interlocking protrusion, wherein the base comprises an interlocking recess, and wherein the interlocking protrusion and the interlocking recess provide a means for the interlocking of the lid and the base. In various embodiments, the IOL holder includes an integral latching mechanism that automatically interlocks and holds the lid and the base in a closed position, wherein the engagement of the latching mechanism can be reversed. In various embodiments, the IOL holder includes an interlocking latch on the lid and an integral post on the base that provide a means for the reversible interlocking of the lid and the base. In various embodiments, the IOL holder includes an interlocking latch on the base and an integral post on the lid that provide a means for the reversible interlocking of the lid and the base.

The reversibility of the interlocking of the lid and the base is meant to require the application of manual force approximating the manual force that can be generated by an adult human hand while the IOL holder is being held by such adult human. In the embodiment of the invention depicted in FIGS. 1 and 3, the lid 12 of the IOL holder comprises an interlocking recess 21, the base 11 comprises an interlocking protrusion 22, and when the IOL holder is in the closed position, the interlocking recess 21 and the interlocking protrusion 22 provide a means for the automatic and reversible interlocking of the lid and the base.

FIG. 1 illustrates an exemplary IOL holder disclosed herein displayed with a lid 12 in an open position and with a single piece intraocular lens 24 in place on the integral annular support 14 with the haptics 23 in contact with the recess features 18 of the integral posts 17. This exemplary IOL holder comprises a one-piece device comprising a base 11, a lid 12, and two integral hinges 13 that connect the base 11 to the lid 12, an integral annular support 14 protruding from the base 11, an integral adaptive feature 15 of a cantilever design that is connected at one end to the lid 12, an integral annular protrusion 16 protruding from the integral adaptive feature 15, two integral posts 17 that each include a recess feature 18, circular apertures 19 in the lid 12, rectangular apertures 20 in the base 11, and a means for interlocking the lid 12 and the base 11 including an interlocking recess 21 in the lid 12 and an interlocking protrusion 22 in the base 11.

FIG. 2 illustrates the same exemplary IOL holder illustrated in FIG. 1 displayed with the lid 12 in an open position with a multiple piece (3-piece) intraocular lens in place on the integral annular support 14 protruding from the base 11, and the haptics 23 partially wrapped around the integral posts 17.

FIG. 3 illustrates the same exemplary IOL holder illustrated in FIG. 1 displayed with the lid 12 in a closed position upon the base 11.

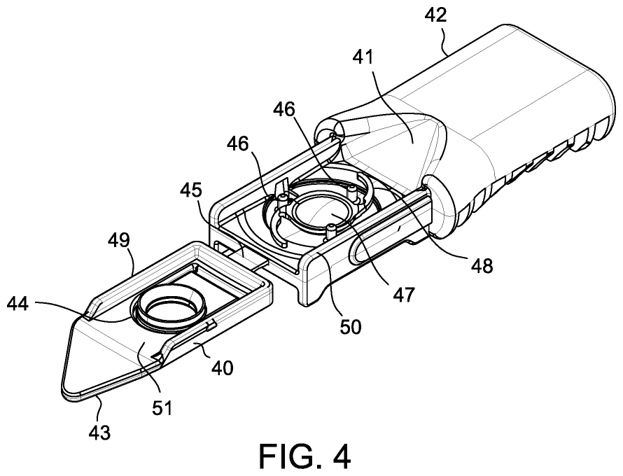

FIG. 4 illustrates an exemplary IOL holder disclosed herein displayed with a lid 40 in an open position and with a single piece intraocular lens 47 resting on the floor of the IOL holder body 42 with the haptics 48 in contact with the retention posts 46. The retention posts 46 may be in contact the body of the intraocular lens 47 to suitably hold the intraocular lens 47 in a static position within the body 42 when lid 40 is in the open position. This exemplary IOL holder comprises a one-piece device comprising a body 42, a lid 40, and an integral hinge 45 that connect the body 42 to the lid 40, an integral adaptive feature 51 of a cantilever design that is connected at one end to the lid 40, an integral annular protrusion 44 protruding from the integral adaptive feature 51, three retention posts 46, and a means for the lid 40 and the body 42 including an interlocking sides 49 in the lid 40 and an interlocking protrusion 50 in the body 42. The body 42 includes a recess 41 which allows tab 43 of the lid 40 to at least partially contact body 42.

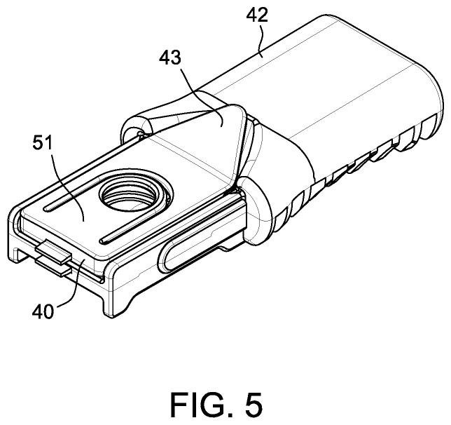

FIG. 5 illustrates the same exemplary IOL holder illustrated in FIG. 4 displayed with the lid 40 in a closed position upon the body 42.

Various embodiments of the disclosure further relate to a method of manufacture of an IOL holder comprising forming an IOL holder from at least one polymer, wherein the IOL holder comprises a one-piece device comprising a base, a lid, and at least one integral hinge that connects the base to the lid, wherein the base of the IOL holder comprises an integral annular support that is designed to accommodate an intraocular lens, and wherein the lid of the IOL holder comprises an integral adaptive feature that exerts a force on a lens body of an intraocular lens when an intraocular lens is positioned on the integral annular support of the base and the lid is in a closed position.

In various embodiments, the method of manufacture of an IOL holder comprises forming an IOL holder from at least one polymer, wherein the at least one polymer is molded. In various embodiments, the method of manufacture of an IOL holder comprises forming an IOL holder from at least one polymer, wherein the at least one polymer is printed. Suitable polymers include those that are compatible with the material of construction of an intraocular lens and the methods of use of the IOL holder including, but not limited to, thermal plastics, biopolymers, and copolymers. More specifically, the polymers include, but are not limited to, polyolefins (e.g., polypropylene and polyethylene), polycarbonate, acrylic polymers, acrylic copolymers, nylon, polyester, cellulose acetate, acetate/butyrate, and the like. Preferably, the method of manufacture comprises molding or printing the IOL holder from polypropylene.

In various embodiments, the method of manufacture of an IOL holder comprises forming an IOL holder that can accommodate various types, models, shapes, and sizes of a single piece or a multiple piece intraocular lens.

In various embodiments, the method of manufacture of an IOL holder comprises forming an IOL holder that includes an integral annular support that is designed to protrude from the base of the IOL holder as a column comprising a concave profile at the distal end of the integral annular support with respect to the base. In various embodiments, the method of manufacture of an IOL holder comprises forming an IOL holder that includes an integral annular support that is designed to be level with the base of the IOL holder and comprise a concave profile. In various embodiments, the method of manufacture of an IOL holder comprises forming an IOL holder that includes an integral annular support that is designed to recede within the base, of the IOL holder and comprise a concave profile. Preferably, the method of manufacture comprises forming an IOL holder that includes an integral annular support that protrudes from the base in a columnar shape and comprises a concave profile at the distal end of the integral annular support with respect to the base.

In various embodiments, the method of manufacture of an IOL holder comprises forming an IOL holder that includes an integral adaptive feature that comprises a cantilever design, wherein only one end of the integral adaptive feature is integrally connected to the lid. In various embodiments, the method of manufacture of an IOL holder comprises forming an IOL holder that includes an integral adaptive feature that comprises a cantilever design, wherein the integral adaptive feature is capable of being positioned at angles inward from, outward from, and/or level with the lid along the length of the integral adaptive feature.

In various embodiments, the method of manufacture of an IOL holder comprises forming an IOL holder that includes an angle of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of manufacture of an IOL holder comprises forming an IOL holder that includes a length of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of manufacture of an IOL holder comprises forming an IOL holder that includes a width of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of manufacture of an IOL holder comprises forming an IOL holder that includes a thickness of the of the integral connection of the integral adaptive feature with to the lid that is set during manufacturing such that the integral adaptive feature will exert a desired force on a lens body of an intraocular lens when an intraocular lens is positioned on the integral annular support of the base and a lid is in a closed position.

In various embodiments, the method of manufacture of an IOL holder comprises forming an IOL holder that includes an integral adaptive feature that comprises an integral annular protrusion that exerts a force on a lens body of an intraocular lens when an intraocular lens is positioned on the integral annular support of the base and a lid is in a closed position. In various embodiments, the method of manufacture of an IOL holder comprises forming an IOL holder that includes an integral adaptive feature that comprises an integral annular protrusion that comprises a concave profile and is positioned to apply a desired force on an intraocular lens when an intraocular lens is placed in the integrated annular support of the base and the lid is in a closed position. A desired force in this situation is one that would generally maintain the intraocular lens in position, but would not deform or damage the intraocular lens when the intraocular lens is placed in the integrated annular support of the base and the lid is in a closed position.

In various embodiments, the method of manufacture of an IOL holder comprises forming an IOL holder that includes an integral adaptive feature that comprises an integral annular recession that exerts a force on a lens body of an intraocular lens when an intraocular lens is positioned on the integral annular support of the base and a lid is in a closed position. In various embodiments, the method of manufacture of an IOL holder comprises forming an IOL holder that includes an integral adaptive feature that comprises an integral annular recession that comprises a concave profile and is positioned to apply a desired force on an intraocular lens when an intraocular lens is placed in the integrated annular support of the base and the lid is in a closed position.

In various embodiments, the method of manufacture of an IOL holder comprises forming an IOL holder that includes an integral adaptive feature that comprises an integral annular feature that is level with the lid and that exerts a force on a lens body of an intraocular lens when an intraocular lens is positioned on the integral annular support of the base and a lid is in a closed position. In various embodiments, the method of manufacture of an IOL holder comprises forming an IOL holder that includes an integral adaptive feature that comprises an integral annular feature that is level with the lid and that comprises a concave profile and is positioned to apply a desired force on an intraocular lens when an intraocular lens is placed in the integrated annular support of the base and the lid is in a closed position.

In various embodiments, the method of manufacture of an IOL holder comprises forming an IOL holder that includes an integral adaptive feature that exerts a force on a lens body of an intraocular lens when an intraocular lens is positioned on the integral annular support of the base and a lid is in a closed position, wherein the force is enough to hold an intraocular lens in place on an integral annular support of a base without causing damage to an intraocular lens.

Preferably, the method of manufacture of an IOL holder comprises forming an IOL holder that includes an integral adaptive feature that comprises an integral annular protrusion that applies a desired force on an intraocular lens when an intraocular lens is placed in the integrated annular support of the base and the lid is in a closed position such that the force is enough to hold an intraocular lens in place on an integral annular support of a base without causing damage to an intraocular lens.

In various embodiments, the method of manufacture of an IOL holder comprises forming an IOL holder that includes a base that comprises one or more integral posts positioned in proximity to an integral annular support of the base. In various embodiments, the method of manufacture of an IOL holder comprises forming an IOL holder that includes one or more integral posts positioned at a distance from the integral annular support such that the haptics of an intraocular lens placed on the integrated annular support can make contact with the one or more integral posts.

In various embodiments, the method of manufacture of an IOL holder comprises forming an IOL holder that includes a base that comprises one or more integral posts positioned in proximity to an integral annular support of the base, wherein the one or more integral posts provide one or more contact points for one or more haptics when an intraocular lens is positioned on the integral annular support of the base. In various embodiments, the method of manufacture of an IOL holder comprises forming an IOL holder wherein the haptics of an intraocular lens placed on the integrated annular support can make contact with the top of the one or more integral posts. In various embodiments, the method of manufacture of an IOL holder comprises forming an IOL holder wherein the haptics of an intraocular lens placed on the integrated annular support can make contact with the sides of the one or more integral posts. In various embodiments, the method of manufacture of an IOL holder comprises forming an IOL holder wherein the haptics of an intraocular lens placed on the integrated annular support can wrap at least partially around the sides of the one or more integral posts.

In various embodiments, the method of manufacture of an IOL holder comprises forming an IOL holder that includes a base that comprises one or more integral posts positioned in proximity to an integral annular support of the base, wherein the one or more integral posts comprise at least one recess feature. In various embodiments, the method of manufacture of an IOL holder comprises forming an IOL holder wherein the at least one recess feature is positioned on the top of the one or more integral posts. In various embodiments, the method of manufacture of an IOL holder comprises forming an IOL holder wherein the at least one recess feature is positioned on the side of the one or more integral posts.

In various embodiments, the method of manufacture of an IOL holder comprises forming an IOL holder that includes a base that comprises one or more integral posts positioned in proximity to an integral annular support of the base, wherein the one or more integral posts comprise at least one recess feature, wherein the at least one recess feature of the one or more integral posts provides one or more contact points for one or more haptics when an intraocular lens is positioned on the integral annular support of the base. In various embodiments, the method of manufacture of an IOL holder comprises forming an IOL holder wherein the haptics of an intraocular lens placed on the integrated annular support can make contact with the at least one recess feature that is positioned on the top of the one or more integral posts. In various embodiments, the method of manufacture of an IOL holder comprises forming an IOL holder wherein the haptics of an intraocular lens placed on the integrated annular support can make contact with the at least one recess feature that is positioned on the side of the one or more integral posts. In various embodiments, the method of manufacture of an IOL holder comprises forming an IOL holder wherein the haptics of an intraocular lens placed on the integrated annular support can make contact with the at least one recess feature that is positioned on the side of the one or more integral posts when the haptics wrap at least partially around the sides of the one or more integral posts.

Preferentially, the method of manufacture of an IOL holder comprises forming an IOL holder wherein the base of the IOL holder comprises two integral posts that each include at least one recess feature, wherein the integral posts are positioned at a distance from the integral annular support of the base such that the haptics of a single piece intraocular lens placed on the integrated annular support can make contact with the at least one recess feature that is positioned on the top of each of the integral posts, and wherein the haptics of a three piece intraocular lens placed on the integrated annular support can at least partially wrap around and contact the sides of the integral posts.

In various embodiments, the method of manufacture of an IOL holder comprises forming an IOL holder that includes a lid that comprises one or more apertures to allow gas and/or liquid to flow through to an intraocular lens when an intraocular lens is positioned on the integral annular support of the base and the lid is in the closed position. In various embodiments, the method of manufacture of an IOL holder comprises forming an IOL holder wherein the one or more apertures included in the lid are arranged in a semicircular pattern positioned around the portion of the lid that is in close proximity to the end of the integral adaptive feature that exerts a force on a lens body of an intraocular lens when an intraocular lens is positioned on the integral annular support of the base and the lid is in a closed position. In various embodiments, the method of manufacture of an IOL holder comprises forming an IOL holder wherein the one or more apertures included in the lid are arranged in a linear pattern. In various embodiments, the method of manufacture of an IOL holder comprises forming an IOL holder wherein the one or more apertures included in the lid are arranged in a partial square or rectangular pattern. In various embodiments, the method of manufacture of an IOL holder comprises forming an IOL holder wherein the one or more apertures included in the lid are each circular, ovular, square, rectangular, or triangular in shape. Preferentially, the method of manufacture of an IOL holder comprises forming an IOL holder wherein the one or more apertures included in the lid are circular in shape and arranged in a semicircular pattern.

In various embodiments, the method of manufacture of an IOL holder comprises forming an IOL holder that includes a base that comprises one or more apertures to allow gas and/or liquid to flow through to an intraocular lens when an intraocular lens is positioned on the integral annular support of the base and the lid is in the closed position. In various embodiments, the method of manufacture of an IOL holder comprises forming an IOL holder wherein the one or more apertures included in the base are arranged in a linear pattern aligned from the end distal to the integral annular support and leading towards, but short of the integral annular support. In various embodiments, the method of manufacture of an IOL holder comprises forming an IOL holder wherein the one or more apertures included in the base are arranged in a diagonal pattern. In various embodiments, the method of manufacture of an IOL holder comprises forming an IOL holder wherein the one or more apertures included in the base are arranged in a circular, ovular, square, rectangular, or triangular pattern. In various embodiments, the method of manufacture of an IOL holder comprises forming an IOL holder wherein the one or more apertures included in the base are each circular, ovular, square, rectangular, or triangular in shape. Preferentially, the method of manufacture of an IOL holder comprises forming an IOL holder wherein the one or more apertures included in the base are rectangular in shape and arranged in a linear pattern.

In various embodiments, the method of manufacture of an IOL holder comprises forming an IOL holder that includes a lid that automatically interlocks with a base when the lid is in the closed position, wherein the interlocking of the lid and the base is reversible. In various embodiments, the method of manufacture of an IOL holder comprises forming an IOL holder that includes a lid that automatically interlocks with a base when the lid is in the closed position, wherein the interlocking of the lid and the base is reversible, wherein the lid comprises an interlocking recess, wherein the base comprises an interlocking protrusion, and wherein the interlocking recess and the interlocking protrusion provide a means for the interlocking of the lid and the base. In various embodiments, the method of manufacture of an IOL holder comprises forming an IOL holder that includes a lid that automatically interlocks with a base when the lid is in the closed position, wherein the interlocking of the lid and the base is reversible, wherein the lid comprises an interlocking protrusion, wherein the base comprises an interlocking recess, and wherein the interlocking protrusion and the interlocking recess provide a means for the interlocking of the lid and the base. In various embodiments, the method of manufacture of an IOL holder comprises forming an IOL holder that includes an integral latching mechanism that automatically interlocks and holds the lid and the base in a closed position, wherein the engagement of the latching mechanism can be reversed. In various embodiments, the method of manufacture of an IOL holder comprises forming an IOL holder that includes an interlocking latch on the lid and an integral post on the base that provide a means for the reversible interlocking of the lid and the base. In various embodiments, the method of manufacture of an IOL holder comprises forming an IOL holder that includes an interlocking latch on the base and an integral post on the lid that provide a means for the reversible interlocking of the lid and the base.

The reversibility of the interlocking of the lid and the base is meant to require the application of manual force approximating the manual force that can be generated by an adult human hand while the IOL holder is being held by such adult human. Preferentially, the method of manufacture of an IOL holder comprises forming an IOL holder wherein the lid of the IOL holder comprises an interlocking recess, the base comprises an interlocking protrusion, and when the IOL holder is in the closed position, the interlocking recess and the interlocking protrusion provide a means for the automatic and reversible interlocking of the lid and the base.

Various embodiments of the disclosure further relate to a method of use of an IOL holder comprising holding, transporting, shipping, carrying, washing, sterilizing, aerating, degassing, and/or storing an intraocular lens in the IOL holder, wherein the IOL holder comprises a one-piece device comprising a base, a lid, and at least one integral hinge that connects the base to the lid, wherein the base of the IOL holder comprises an integral annular support that is designed to accommodate an intraocular lens, and wherein the lid of the IOL holder comprises an integral adaptive feature that exerts a force on a lens body of an intraocular lens when an intraocular lens is positioned on the integral annular support of the base and the lid is in a closed position.

In various embodiments, the method of use comprises holding and/or storing an intraocular lens in the IOL holder, wherein the intraocular lens is held and/or stored in a substantially unfolded state such that the haptics are substantially in their natural position with respect to the optic. In various embodiments, the method of use comprises holding and/or storing an intraocular lens in the IOL holder, wherein the intraocular lens is held and/or stored in a partially folded state such that the haptics are partially folded towards, above, and/or below the optic. In various embodiments, the method of use comprises holding and/or storing an intraocular lens in the IOL holder, wherein the intraocular lens is held and/or stored in a substantially folded state such that the haptics are substantially folded towards, above, and/or below the optic. Preferentially, the IOL holder holds an intraocular lens in a substantially unfolded state.

In various embodiments, the method of use comprises washing, sterilizing, aerating, and/or degassing an intraocular lens in the IOL holder, wherein the intraocular lens is substantially washed, sterilized, aerated, and/or degassed using techniques and procedures that are known to be compatible with an intraocular lens. In various embodiments, the method of use comprises transporting, shipping, and/or carrying an intraocular lens in the IOL holder, wherein the intraocular lens is transported, shipped, and/or carried using techniques and procedures that are known to be compatible with an intraocular lens.

In various embodiments, the method of use of an IOL holder comprises using an IOL holder that is formed from at least one polymer, wherein the at least one polymer is molded. In various embodiments, the method of use of an IOL holder comprises using an IOL holder that is formed from at least one polymer, wherein the at least one polymer is printed. Suitable polymers include those that are compatible with the material of construction of an intraocular lens and the methods of use of the IOL holder including, but not limited to, thermal plastics, biopolymers, and copolymers. More specifically, the polymers include, but are not limited to, polyolefins (e.g., polypropylene and polyethylene), polycarbonate, acrylic polymers, acrylic copolymers, nylon, polyester, cellulose acetate, acetate/butyrate, and the like. Preferably, the method of manufacture comprises molding or printing the IOL holder from polypropylene.

In various embodiments, the method of use of an IOL holder comprises using an IOL holder that can accommodate various types, models, shapes, and sizes of a single piece or a multiple piece intraocular lens.

In various embodiments, the method of use of an IOL holder comprises using an IOL holder that includes an integral annular support that is designed to protrude from the base of the IOL holder as a column comprising a concave profile at the distal end of the integral annular support with respect to the base. In various embodiments, the method of use of an IOL holder comprises using an IOL holder that includes an integral annular support that is designed to be level with the base of the IOL holder and comprise a concave profile. In various embodiments, the method of use of an IOL holder comprises using an IOL holder that includes an integral annular support that is designed to recede within the base, of the IOL holder and comprise a concave profile. Preferably, the method of use of an IOL holder comprises using an IOL holder that includes an integral annular support that protrudes from the base in a columnar shape and comprises a concave profile at the distal end of the integral annular support with respect to the base.

In various embodiments, the method of use of an IOL holder comprises using an IOL holder that includes an integral adaptive feature that comprises a cantilever design, wherein only one end of the integral adaptive feature is integrally connected to the lid. In various embodiments, the method of use of an IOL holder comprises using an IOL holder that includes an integral adaptive feature that comprises a cantilever design, wherein the integral adaptive feature is capable of being positioned at angles inward from, outward from, and/or level with the lid along the length of the integral adaptive feature.

In various embodiments, the method of use of an IOL holder comprises using an IOL holder that includes an angle of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of use of an IOL holder comprises using an IOL holder that includes a length of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of use of an IOL holder comprises using an IOL holder that includes a width of the integral adaptive feature with respect to the lid that is set during manufacturing such that the integral adaptive feature will exert a desired force on a lens body of an intraocular lens when an intraocular lens is positioned on the integral annular support of the base and a lid is in a closed position. In various embodiments, the method of use of an IOL holder comprises using an IOL holder that includes a thickness of the of the integral connection of the integral adaptive feature with to the lid that is set during manufacturing such that the integral adaptive feature will exert a desired force on a lens body of an intraocular lens when an intraocular lens is positioned on the integral annular support of the base and a lid is in a closed position.

In various embodiments, the method of use of an IOL holder comprises using an IOL holder that includes an integral adaptive feature that comprises an integral annular protrusion that exerts a force on a lens body of an intraocular lens when an intraocular lens is positioned on the integral annular support of the base and a lid is in a closed position. In various embodiments, the method of use of an IOL holder comprises using an IOL holder that includes an integral adaptive feature that comprises an integral annular protrusion that comprises a concave profile and is positioned to apply a desired force on an intraocular lens when an intraocular lens is placed in the integrated annular support of the base and the lid is in a closed position.

In various embodiments, the method of use of an IOL holder comprises using an IOL holder that includes an integral adaptive feature that comprises an integral annular recession that exerts a force on a lens body of an intraocular lens when an intraocular lens is positioned on the integral annular support of the base and a lid is in a closed position. In various embodiments, the method of use of an IOL holder comprises using an IOL holder that includes an integral adaptive feature that comprises an integral annular recession that comprises a concave profile and is positioned to apply a desired force on an intraocular lens when an intraocular lens is placed in the integrated annular support of the base and the lid is in a closed position.

In various embodiments, the method of use of an IOL holder comprises using an IOL holder that includes an integral adaptive feature that comprises an integral annular feature that is level with the lid and that exerts a force on a lens body of an intraocular lens when an intraocular lens is positioned on the integral annular support of the base and a lid is in a closed position. In various embodiments, the method of use of an IOL holder comprises using an IOL holder that includes an integral adaptive feature that comprises an integral annular feature that is level with the lid and that comprises a concave profile and is positioned to apply a desired force on an intraocular lens when an intraocular lens is placed in the integrated annular support of the base and the lid is in a closed position.

In various embodiments, the method of use of an IOL holder comprises using an IOL holder that includes an integral adaptive feature that exerts a force on a lens body of an intraocular lens when an intraocular lens is positioned on the integral annular support of the base and a lid is in a closed position, wherein the force is enough to hold an intraocular lens in place on an integral annular support of a base without causing damage to an intraocular lens.

Preferably, the method of use of an IOL holder comprises using an IOL holder that includes an integral adaptive feature that comprises an integral annular protrusion that applies a desired force on an intraocular lens when an intraocular lens is placed in the integrated annular support of the base and the lid is in a closed position such that the force is enough to hold an intraocular lens in place on an integral annular support of a base without causing damage to an intraocular lens.

In various embodiments, the method of use of an IOL holder comprises using an IOL holder that includes a base that comprises one or more integral posts positioned in proximity to an integral annular support of the base. In various embodiments, the method of use of an IOL holder comprises using an IOL holder that includes one or more integral posts positioned at a distance from the integral annular support such that the haptics of an intraocular lens placed on the integrated annular support can make contact with the one or more integral posts.

In various embodiments, the method of use of an IOL holder comprises using an IOL holder that includes a base that comprises one or more integral posts positioned in proximity to an integral annular support of the base, wherein the one or more integral posts provide one or more contact points for one or more haptics when an intraocular lens is positioned on the integral annular support of the base. In various embodiments, the method of use of an IOL holder comprises using an IOL holder wherein the haptics of an intraocular lens placed on the integrated annular support can make contact with the top of the one or more integral posts. In various embodiments, the method of use of an IOL holder comprises using an IOL holder wherein the haptics of an intraocular lens placed on the integrated annular support can make contact with the sides of the one or more integral posts. In various embodiments, the method of use of an IOL holder comprises using an IOL holder wherein the haptics of an intraocular lens placed on the integrated annular support can wrap at least partially around the sides of the one or more integral posts.

In various embodiments, the method of use of an IOL holder comprises using an IOL holder that includes a base that comprises one or more integral posts positioned in proximity to an integral annular support of the base, wherein the one or more integral posts comprise at least one recess feature. In various embodiments, the method of use of an IOL holder comprises using an IOL holder wherein the at least one recess feature is positioned on the top of the one or more integral posts. In various embodiments, the method of use of an IOL holder comprises using an IOL holder wherein the at least one recess feature is positioned on the side of the one or more integral posts.

In various embodiments, the method of use of an IOL holder comprises using an IOL holder that includes a base that comprises one or more integral posts positioned in proximity to an integral annular support of the base, wherein the one or more integral posts comprise at least one recess feature, wherein the at least one recess feature of the one or more integral posts provides one or more contact points for one or more haptics when an intraocular lens is positioned on the integral annular support of the base. In various embodiments, the method of use of an IOL holder comprises using an IOL holder wherein the haptics of an intraocular lens placed on the integrated annular support can make contact with the at least one recess feature that is positioned on the top of the one or more integral posts. In various embodiments, the method of use of an IOL holder comprises using an IOL holder wherein the haptics of an intraocular lens placed on the integrated annular support can make contact with the at least one recess feature that is positioned on the side of the one or more integral posts. In various embodiments, the method of use of an IOL holder comprises using an IOL holder wherein the haptics of an intraocular lens placed on the integrated annular support can make contact with the at least one recess feature that is positioned on the side of the one or more integral posts when the haptics wrap at least partially around the sides of the one or more integral posts.

Preferentially, the method of use of an IOL holder comprises using an IOL holder wherein the base of the IOL holder comprises two integral posts that each include at least one recess feature, wherein the integral posts are positioned at a distance from the integral annular support of the base such that the haptics of a single piece intraocular lens placed on the integrated annular support can make contact with the at least one recess feature that is positioned on the top of each of the integral posts, and wherein the haptics of a three piece intraocular lens placed on the integrated annular support can at least partially wrap around and contact the sides of the integral posts.

In various embodiments, the method of use of an IOL holder comprises using an IOL holder that includes a lid that comprises one or more apertures to allow gas and/or liquid to flow through to an intraocular lens when an intraocular lens is positioned on the integral annular support of the base and the lid is in the closed position. In various embodiments, the method of use of an IOL holder comprises using an IOL holder wherein the one or more apertures included in the lid are arranged in a semicircular pattern positioned around the portion of the lid that is in close proximity to the end of the integral adaptive feature that exerts a force on a lens body of an intraocular lens when an intraocular lens is positioned on the integral annular support of the base and the lid is in a closed position. In various embodiments, the method of use of an IOL holder comprises using an IOL holder wherein the one or more apertures included in the lid are arranged in a linear pattern. In various embodiments, the method of use of an IOL holder comprises using an IOL holder wherein the one or more apertures included in the lid are arranged in a partial square or rectangular pattern. In various embodiments, the method of use of an IOL holder comprises using an IOL holder wherein the one or more apertures included in the lid are each circular, ovular, square, rectangular, or triangular in shape. Preferentially, the method of use of an IOL holder comprises using an IOL holder wherein the one or more apertures included in the lid are circular in shape and arranged in a semicircular pattern.

In various embodiments, the method of use of an IOL holder comprises using an IOL holder that includes a base that comprises one or more apertures to allow gas and/or liquid to flow through to an intraocular lens when an intraocular lens is positioned on the integral annular support of the base and the lid is in the closed position. In various embodiments, the method of use of an IOL holder comprises using an IOL holder wherein the one or more apertures included in the base are arranged in a linear pattern aligned from the end distal to the integral annular support and leading towards, but short of the integral annular support. In various embodiments, the method of use of an IOL holder comprises using an IOL holder wherein the one or more apertures included in the base are arranged in a diagonal pattern. In various embodiments, the method of use of an IOL holder comprises using an IOL holder wherein the one or more apertures included in the base are arranged in a circular, ovular, square, rectangular, or triangular pattern. In various embodiments, the method of use of an IOL holder comprises using an IOL holder wherein the one or more apertures included in the base are each circular, ovular, square, rectangular, or triangular in shape. Preferentially, the method of use of an IOL holder comprises using an IOL holder wherein the one or more apertures included in the base are rectangular in shape and arranged in a linear pattern.

In various embodiments, the method of use of an IOL holder comprises using an IOL holder that includes a lid that automatically interlocks with a base when the lid is in the closed position, wherein the interlocking of the lid and the base is reversible. In various embodiments, the method of use of an IOL holder comprises using an IOL holder that includes a lid that automatically interlocks with a base when the lid is in the closed position, wherein the interlocking of the lid and the base is reversible, wherein the lid comprises an interlocking recess, wherein the base comprises an interlocking protrusion, and wherein the interlocking recess and the interlocking protrusion provide a means for the interlocking of the lid and the base. In various embodiments, the method of use of an IOL holder comprises using an IOL holder that includes a lid that automatically interlocks with a base when the lid is in the closed position, wherein the interlocking of the lid and the base is reversible, wherein the lid comprises an interlocking protrusion, wherein the base comprises an interlocking recess, and wherein the interlocking protrusion and the interlocking recess provide a means for the interlocking of the lid and the base. In various embodiments, the method of use of an IOL holder comprises using an IOL holder that includes an integral latching mechanism that automatically interlocks and holds the lid and the base in a closed position, wherein the engagement of the latching mechanism can be reversed. In various embodiments, the method of use of an IOL holder comprises using an IOL holder that includes an interlocking latch on the lid and an integral post on the base that provide a means for the reversible interlocking of the lid and the base. In various embodiments, the method of use of an IOL holder comprises using an IOL holder that includes an interlocking latch on the base and an integral post on the lid that provide a means for the reversible interlocking of the lid and the base.

The reversibility of the interlocking of the lid and the base is meant to require the application of manual force approximating the manual force that can be generated by an adult human hand while the IOL holder is being held by such adult human. Preferentially, the method of use of an IOL holder comprises using an IOL holder wherein the lid of the IOL holder comprises an interlocking recess, the base comprises an interlocking protrusion, and when the IOL holder is in the closed position, the interlocking recess and the interlocking protrusion provide a means for the automatic and reversible interlocking of the lid and the base.

1. A one-piece intraocular lens (IOL) holder comprising a first portion, a second portion, and at least one integral hinge connecting the first and second portions, the holder having a first, open position, and a second, closed position,
wherein the first portion comprises an integral annular support configured to accommodate a lens body of an intraocular lens, and
wherein the second portion comprises an integral adaptive feature configured to exert a force on the intraocular lens positioned on the integral annular support of the first portion when the holder in in the second position.

2. The IOL holder according to claim 1, wherein the integral adaptive feature is configured to exert a force on the lens body of the intraocular lens when the holder in in the second position.

3. The IOL holder according to claim 1, wherein the integral adaptive feature is configured to exert a force on a haptic portion of the intraocular lens when the holder in in the second position.

4. The IOL holder according to claim 1, further comprising an intraocular lens positioned on the integral annular support.

5. The IOL holder of claim 1, wherein the integral adaptive feature comprises a cantilever design, wherein only one portion of the integral adaptive feature is integrally connected to the second portion.

6. The IOL holder of claim 1, wherein the integral adaptive feature is connected to the second portion by a flexible connection.

7. The IOL holder of claim 1, wherein the integral adaptive feature comprises an integral annular protrusion that exerts the force on a lens body of an intraocular lens when an intraocular lens is positioned on the integral annular support and opposing one of the top portion or lower portion is in a closed position.

8. The IOL holder of claim 1, wherein the force is sufficient to hold an intraocular lens in place on the integral annular support of the base when the holder is in the second position without causing damage to the intraocular lens.

9. The IOL holder of claim 1, wherein the first portion comprises one or more integral posts positioned in proximity to the integral annular support and wherein the one or more integral posts provide one or more contact points for one or more haptics when the intraocular lens is positioned on the integral annular support.

10. The IOL holder of claim 9, wherein the one or more integral posts comprise at least one recess feature.

11. The IOL holder of claim 10, wherein the at least one recess feature of the one or more integral posts provides one or more contact points for one or more haptics when the intraocular lens is positioned on the integral annular support.

12. The IOL holder of claim 1, wherein one of the first portion or second portion comprises one or more apertures to allow gas and/or liquid to pass through when the holder is in the second position.

13. The IOL holder of claim 1, wherein the first portion interlocks with the second portion when in the second position.

14. The IOL holder of claim 13, wherein the interlocking of the first portion and the second portion is reversible.

15. The IOL holder of claim 1, wherein one of the first portion and the second portion comprises an interlocking recess and wherein the other of the first portion and the second portion comprises an interlocking protrusion, and wherein the interlocking recess and the interlocking protrusion provide a means for the interlocking of the first portion and the second portion.

16. The IOL holder of claim 1, wherein one of the first portion and the second portion comprises a first segment of an integrally-formed latch capable of interlocking with a second segment of the integrally-formed latch which is integrally-formed with the other of the first portion and the second portion.

17. A method of manufacture of an IOL holder comprising: forming the IOL holder of claim 1 from at least one polymer.

18. The method of manufacture of claim 15, wherein the at least one polymer is molded.

19. The method of manufacture of claim 15, wherein the at least one polymer is printed.

20. A method of use of an IOL holder comprising: holding, transporting, shipping, carrying, washing, sterilizing, aerating, degassing, and/or storing an intraocular lens in the IOL holder of claim 1.
